(19) United States
(12) Patent Application Publication
(10) Pub. No.: US 2025/0064522 A1
(43) Pub. Date: Feb. 27, 2025

(54) PRE-OPERATIVE PLANNING FOR A MULTI-ARM ROBOTIC SURGICAL SYSTEM

(71) Applicant: SSI IP HOLDINGS INC., Fort Lauderdale, FL (US)

(72) Inventors: Vishwajyoti Pascual SRIVASTAVA, Gurugram, Haryana (IN); Rama Kishna Reddy DYAVA, Gurugram, Haryana (IN); Suraj DWIVEDI, Gurugram, Haryana (IN); Sudhir Prem SRIVASTAVA, Gurugram, Haryana (IN)

(21) Appl. No.: 18/721,998

(22) PCT Filed: Nov. 9, 2023
(86) PCT No.: PCT/IN2023/051035

§ 371 Date: Jun. 20, 2024

(30) Foreign Application Priority Data
Nov. 21, 2022 (IN) 202211066743

(51) Int. Cl. A61B 34/10; A61B 17/00; A61B 34/00; A61B 34/30; G16H 10/60

(52) U.S. Cl. CPC A61B 34/10; A61B 17/00234; A61B 34/25; A61B 34/30; G16H 10/60; A61B 2034/101; A61B 2034/252; A61B 2034/256; A61B 2034/301

(57) ABSTRACT

The application provides a method (700) for pre-operative planning for a multi-arm robotic surgical system (100) comprising surgical instruments (110, 112, 114, 116) coupled to robotic arms (102b, 102c, 102d, 102e), and a camera (C) coupled to robotic arm (102a). Each robotic arm is mounted on one of a plurality of robotic carts (SL, PL, CA, PR, SR) arranged along an operating table (104) and a patient lying on the operating table (104). The method (700) comprises storing (702) at least one of various human anatomical data and body habitus in a database (304) of a server (302). Then, the server (302) creates (704) a set of models indicating placement of ports on the body of the patient. A graphical user interface (GUI) (306) receives (706) the required patient data and details of a surgical procedure to be performed as input. A processor (308) analyzes (708) the received input with respect to the set of models and identifies (710) an appropriate model. The location of each robotic cart is detected (712) and displayed (714). An optimum configuration of each of the robotic arms and a desired location of each of the plurality of robotic carts is calculated (716). The plurality of robotic carts is placed (718) at the desired location of the plurality of robotic carts having the optimum configuration of each of the robotic arm. The distal end of the plurality of robotic arms is manipulated (720) and docked (722) according to the identified optimum placement of ports on the body of the patient.

### TECHNICAL FIELD

The present disclosure generally relates to a multi-arm robotic surgical system for minimally invasive surgery, and more particularly, the disclosure relates to a system and method for pre-operative planning for a multi-arm robotic surgical system.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as an admissions of prior art.

Robotically assisted surgical systems have been adopted worldwide to replace conventional surgical procedures to reduce number of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a hand controller/master controller/surgeon input device at a surgeon console to seamlessly capture and transfer complex actions performed by the surgeon giving the perception that the surgeon is directly articulating surgical tools/surgical instruments to perform the surgery. The surgeon operating on the surgeon console may be located at a distance from a surgical site or may be located within an operating theatre where the patient is being operated.

The robotically assisted surgeries have revolutionized the medical field and are one of the fastest growing sectors in the medical device industry. However, the major challenge in robotically assisted surgeries is to ensure safety and precision during the surgery. One of the key areas of robotically assisted surgeries is the development of surgical robots for minimally invasive surgery. Over the last couple of decades, surgical robots have evolved exponentially and have been a major area of innovation in the medical device industry.

In robotic surgeries, a pre-operative planning and precise placement of the surgical ports is essential so that a surgical tool can comfortably access the workspace at the surgical site. The main challenge with the existing robotically assisted surgical systems is the unavailability of any pre-operative planning technique. Another challenge is that, in the existing multi-arm robotic surgical systems, it is very difficult to determine the optimum location of entry ports on the patient's body. Further, another challenge is that this poor port placement may lead to collision between robotic arms.

In the light of aforementioned challenges, there is a need for a pre-operative planning for a multi-arm robotic surgical system which will solve the above-mentioned problems related to robotic assisted surgeries.

### SUMMARY OF THE DISCLOSURE

Some or all of the above-mentioned problems related to providing training to the surgeons and OT staff are proposed to be addressed by certain embodiments of the present disclosure.

In one aspect, an embodiment of the present disclosure provides a method for pre-operative planning for a multi-arm robotic surgical system comprising a plurality of robotic arms each mounted on one of a plurality of robotic carts, an endoscopic camera coupled to a robotic arm out of the plurality of robotic arms, a plurality of surgical instruments each detachably coupled to a distal end of a robotic arm out of the remaining robotic arms, an operating table, and a patient lying on the operating table, whereby the plurality of robotic carts are arranged along the operating table, the method comprising: storing, using a server, in a database, at least one of various human anatomical data and body habitus; creating, using the server, a set of models indicating placement of ports on the body of the patient, based on the stored human anatomical data and body habitus; receiving, using a graphical user interface, a required patient data and details of a surgical procedure to be performed; analysing, using a processor, the received patient data and details of the surgical procedure to be performed with respect to the set of models; identifying, using the processor, an appropriate model out of the set of models indicating an optimum placement of ports on the body of the patient; detecting, using a tracker, a location of the plurality of robotic carts each having a surgical instrument, with respect to the robotic cart having the camera; displaying, using the graphical user interface, the detected location of the plurality of robotic carts; calculating, using the processor, an optimum configuration of each of the robotic arm and a desired location of each of the plurality of robotic carts, based on the identified model and the detected location of the plurality of robotic carts; placing, the plurality of robotic carts, at the desired location of the plurality of robotic carts having the optimum configuration of each of the robotic arm; manipulating, the distal end of the plurality of robotic arms according to the identified optimum placement of ports on the body of the patient; docking, the plurality of robotic arms; and configuring, the plurality of robotic arms, in the best optimum position with respect to a remote centre of motion.

Optionally, the server comprises at least one of a local database or a cloud-based database.

Optionally, the patient data may include at least one of anatomy of the patient, name, age, sex, body mass index, height etc.

Optionally, the details of a surgical procedure to be performed may include the name of the procedure.

Optionally, the tracker (310) may be any one of an optical tracker, a laser based tracker, or a RFID based tracker, and the like.

Optionally, the tracker can detect the location of the plurality of robotic carts with respect to at least one of a robotic cart having the camera, an operating table, and the like.

Optionally, the tracker sends the detected location of the plurality of robotic carts to the processor.

Optionally, the surgical procedure to be performed may be Prostatectomy, Hysterectomy, Nephrectomy, Coronary artery bypass graft, Mitral valve repair, and the like.

Optionally, the placing of the plurality of robotic carts at the desired location may be done either automatically or manually.

Optionally, the graphical user interface displays surgical instruments which can be used for the surgical procedure to be performed, position of the patient lying on the operating table, and the placement of ports on the body of the patient.

Optionally, the graphical user interface further can display a simulation video based on the received patient data and details of the surgical procedure to be performed.

Optionally, the server is configured to store the patient data and the pre-operative planning for the patient for record keeping and future reference.

Optionally, the patient lying on the operating table can be real or a mannequin used for training purposes.

Optionally, the processor is provided with various parameters to calculate the optimum configuration of each of the robotic arms.

Optionally, the parameters indicate reachability of the plurality of robotic arms, limits of configuration of the plurality of robotic arms, singularities of the plurality of robotic arms, dexterity of the plurality of robotic arms, manipulability of the plurality of robotic arms, and robustness of the plurality of robotic arms.

Other embodiments, systems, methods, apparatus aspects, and features of the invention will become apparent to those skilled in the art from the following detailed description, the accompanying drawings, and the appended claims. It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to the scale. Wherever possible, like elements have been indicated by identical numbers.

Embodiments of the present disclosure will now be described, by way of example only, with reference to the following diagrams wherein:

FIG. 1 illustrates an example implementation of a multi arm teleoperated surgical system which can be used with one or more features in accordance with an embodiment of the disclosure;

FIG. 2 illustrates a five-arm configuration of robotic carts arranged around an operating table in a multi-arm teleoperated surgical system in accordance with an embodiment of the disclosure;

FIG. 3 illustrates a cart housing various components for pre-operative planning in accordance with an embodiment of the disclosure;

FIG. 4 illustrates steps for development of a library with various human anatomical data in accordance with an embodiment of the disclosure;

FIG. 5 illustrates steps for optimizing the robotic arm motion and joint configurations in accordance with an embodiment of the disclosure;

FIG. 6 illustrates a framework for multi arm robotic surgical system optimization in accordance with an embodiment of the disclosure;

FIG. 7 illustrates a flow chart with steps of pre-operative planning method in accordance with an embodiment of the disclosure;

FIG. 8 illustrates an exemplary interface of pre-operative GUI for manually entering patient details in accordance with an embodiment of the disclosure;

FIG. 9(a) illustrates an exemplary interface of ports and instrument tab of pre-operative GUI for showing suggested instruments and port placements in accordance with an embodiment of the disclosure;

FIG. 9(b) illustrates interface of cart placement tab of pre-operative GUI for showing suggested cart placement in accordance with an embodiment of the disclosure;

FIG. 9(c) illustrates interface of arm docking tab of pre-operative GUI for showing suggested arm docking in accordance with an embodiment of the disclosure; and

FIG. 9(d) illustrates interface of patient positioning tab of pre-operative GUI for showing suggested patient positioning in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The device, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

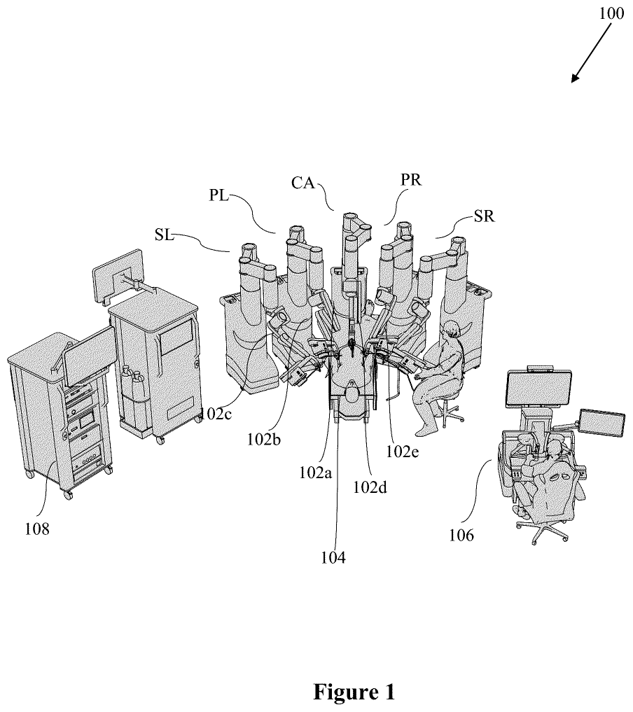

FIG. 1 illustrates an example implementation of a multi arm teleoperated surgical system which can be used with one or more features in accordance with an embodiment of the disclosure. Specifically, FIG. 1 illustrates the multi arm teleoperated surgical system (100) having five robotic arms (102a), (102b), (102c), (102d), (102e), mounted on five robotic arm carts around an operating table (104). The five-robotic arms (102a), (102b), (102c), (102d), (102e), as depicted in FIG. 1, are for illustration purposes and the number of robotic arms may vary depending upon the type of surgery. The exemplary five robotic arms (102a), (102b), (102c), (102d), (102e), are arranged along the operating table (104) and may be arranged in different manner but not limited to the robotic arms (102a), (102b), (102c), (102d), (102e), arranged along the operating table (104). The robotic arms (102a), (102b), (102c), (102d), (102e), may be separately mounted on the five robotic arm carts or the robotic arms (102a), (102b), (102c), (102d), (102e), mechanically and/or operationally connected with each other or the robotic arms (102a), (102b), (102c), (102d), (102e), connected to a central body (not shown) such that the robotic arms (102a), (102b), (102c), (102d), (102e), branch out of a central body (not shown). Further, the multi arm teleoperated surgical system (100) may include a surgeon console (106), a vision cart (108), and a surgical instrument and accessory table.

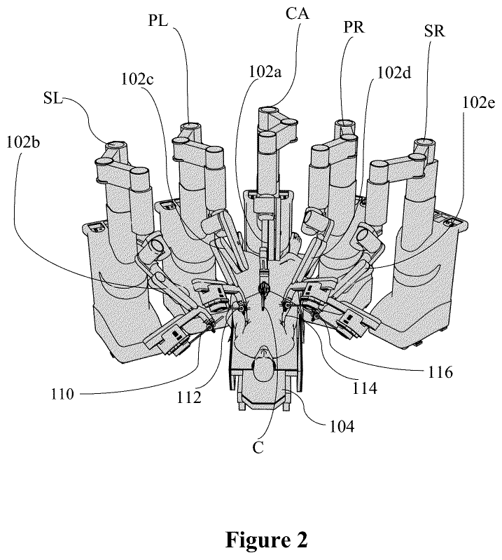

FIG. 2 illustrates a five-arm configuration of robotic carts arranged around an operating table in a multi-arm teleoperated surgical system in accordance with an embodiment of the disclosure. According to an embodiment, the patient side arm carts are indicated as camera arm cart (CA), primary right robotic arm cart (PR), secondary right robotic arm cart (SR), primary left robotic arm cart (PL), and secondary left robotic arm cart (SL). The right and left position of the patient side arm cart carts (PSAC) is named with respect to the surgeon's endoscopic view and not the physical placement of the carts. This is only for identification purposes. An endoscopic camera (C) is coupled to the robotic arm (102a) attached to the camera arm cart (CA). In robotic surgeries, sometimes a surgeon needs to hold a tissue or organ, while performing suturing, clipping, cutting, sealing, and coagulating etc. Then, one robotic arm out of the remaining robotic arms (102b, 102c, 102d, 102e) can be utilized to hold the above-mentioned tissue or organ. Two of the other remaining robotic arms (102b, 102c, 102d, 102e) can be used for other surgical actions. Each of the plurality of surgical instruments (110, 112, 114, 116) is detachably coupled to a robotic arm out of the remaining robotic arms (102b, 102c, 102d, 102e), which in turn is connected to a patient side arm cart out of patient side arm carts (SL, PL, PR, SR).

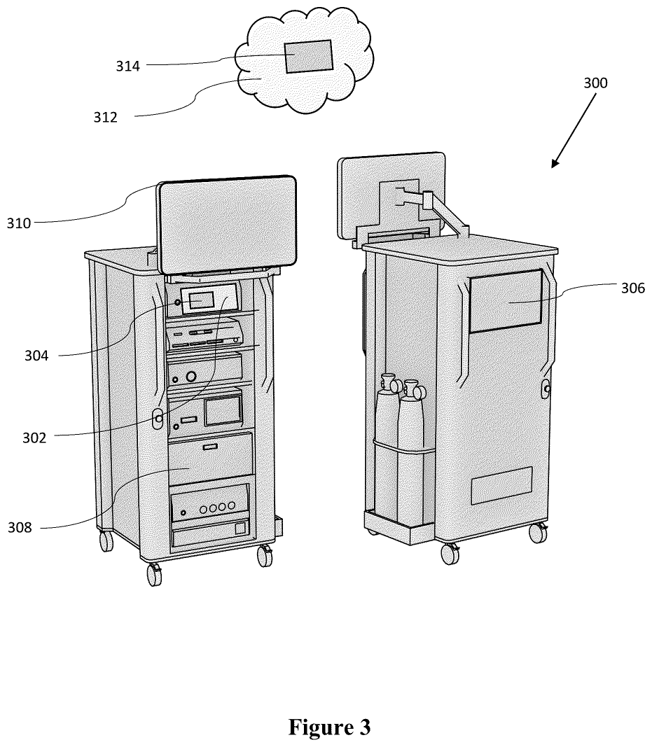

FIG. 3 illustrates a cart (300) housing various components for pre-operative planning in accordance with an embodiment of the disclosure. The cart (300) houses many of the electronic components and controls of the robotic surgical system, like a server (302), a local database (304), a graphical user interface (306), a processor (308) is coupled to the graphical user interface (306), and a tracker (310).

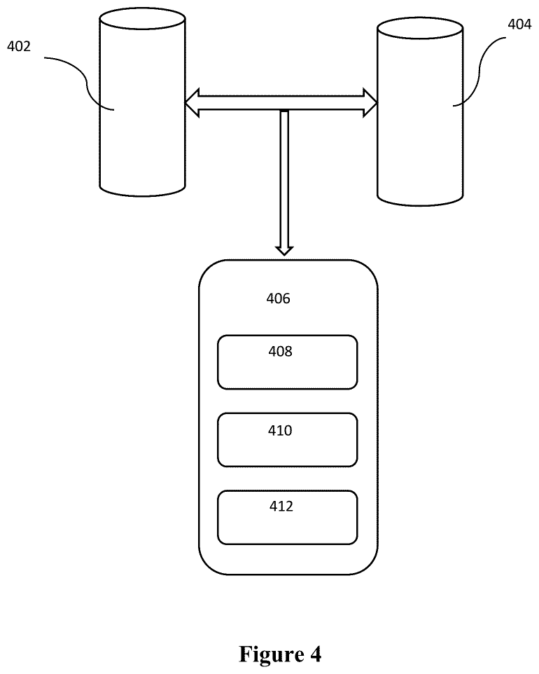

FIG. 4 illustrates steps for development of a library with various human anatomical data in accordance with an embodiment of the disclosure. The entity (402) may depict a database with an anatomical, body mass index (BMI), MRI (magnetic resonance imaging)/CT (computed tomography) data and the like of various human body habitus. Further, the entity (404) may depict data samples for various surgical procedures. The entity (406) may depict a graphical user interface (GUI) which may show a data sample received from the entities (402) and (404). Further, at entity (408), the data samples received from entity (402) and (404) may be processed and at entity (410), the processed data may be compared with various models. Based on the comparison, at entity (412), the optimum model may be predicted which may further predict the optimum port placement.

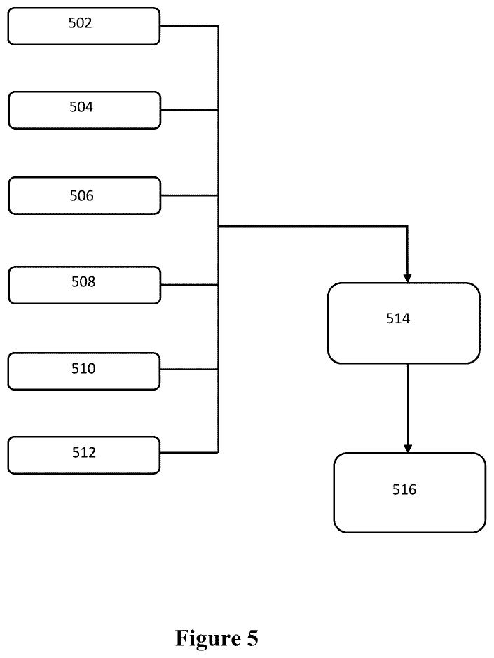

FIG. 5 illustrates steps for optimizing the robotic arm motion and joint configurations in accordance with an embodiment of the disclosure. Various port placement inputs are incorporated to find the optimum joint configurations of the robotic arms. There may be various parameters which may be needed to find the optimum joint configurations of the robotic arms so that there may not be any limitations during the surgical procedure. The parameters are depicted in FIG. 5. At steps (502), the reachability of the arms may be considered. At step (504), joints limits of all the arms may be considered. At step (506), singularities of all the arms may be considered. At step (508), dexterity of all the arms may be considered. At step (510), manipulability of all the arms may be considered. At step (512), robustness index indicating robustness of all the arms may be considered. The aforesaid parameters may be processed at step (514) considering the predicted optimum port placement data and optimum joint positions of all the arms may be predicted at step (516).

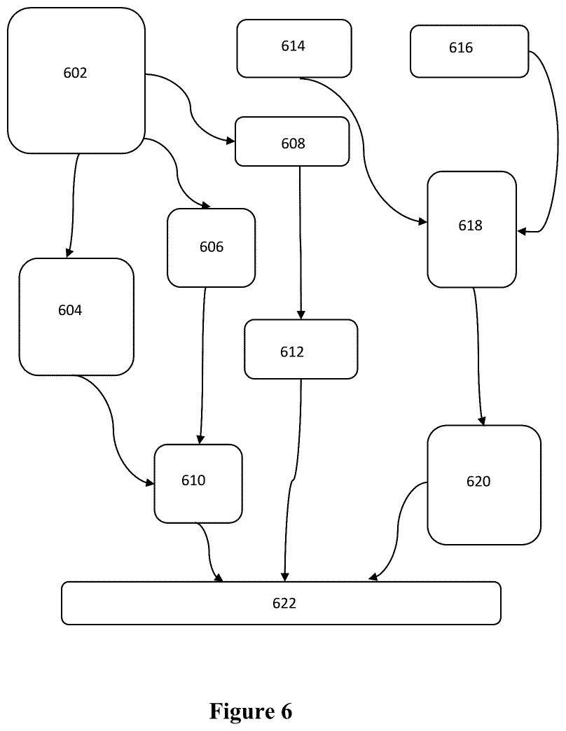

FIG. 6 illustrates a framework for multi arm robotic surgical system optimization in accordance with an embodiment of the disclosure. The entity (602) may depict a multi arm robotic surgical system which may comprise a plurality of robotic arm mounted on carts. For achieving optimization in multi arm robotic surgical system, various parameters may be required but not limited to as described herein. The robotic arm parameters as illustrated in FIG. 5 may be considered as depicted by entity (604). Further, the robotic parameters as depicted at step (604) may be sent to entity (610) which is robustness index and further sent to a multi arm robotic surgical system optimization framework (622).

An entity (606) depicts robot performance index which is sent to the robustness index (610) and further sent to the multi arm robotic surgical system optimization framework (622). An optical tracking system (608) tracks the position of the robotic carts, and the cart registration data (612) is sent to the multi arm robotic surgical system optimization framework (622).

According to an embodiment, a patient data (614) and various surgical procedure data (616) such as gallbladder, Prostatectomy, Hysterectomy, Nephrectomy, Coronary artery bypass graft, Mitral valve repair, and the like, may be sent to entity (618). The entity (618) depicts predicted optimum port placements data based as illustrated previously in FIGS. 4 and 5. Further, the data from entity (618) is sent to an entity (620) which may be approach angles criteria and finally all the data are sent to the multi arm robotic surgical system optimization framework (622).

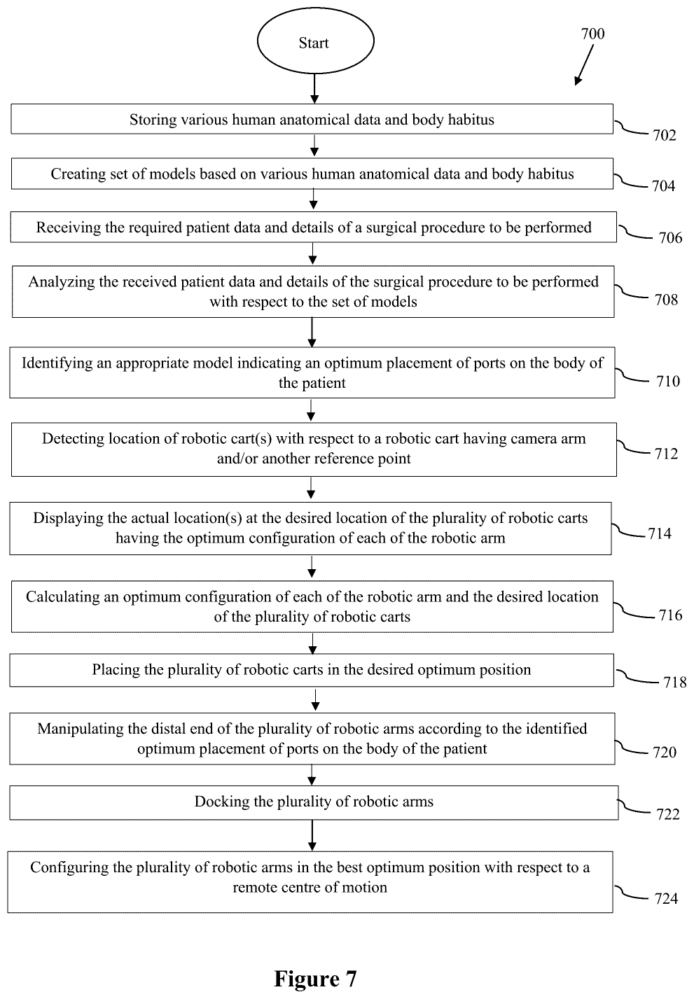

FIG. 7 illustrates a flow chart with steps of pre-operative planning procedure and guidance method (700) in accordance with an embodiment of the disclosure. The server (302) housed in the cart (300) (as shown in FIG. 3), is configured to store a database having at least one of various human anatomical data and body habitus in step (702). Further, the server (302) is configured to create a set of models indicating placement of ports on the body of a patient based on the various human anatomical data and body habitus as shown in step (704), based on the data as stored at the step (702). The server (302) comprises at least one of a local database (304) or a cloud-based database (314). The graphical user interface (306) is provided to input the patient data and the details of the surgical procedure to be performed by an operator. The processor (308) is coupled to the graphical user interface (306). At step (706), the processor (308) receives the required patient data and details of surgical procedure to be performed from the graphical user interface (306). The patient data may be body anatomical data, body habitus, BMI, CT/MRI, and the like data are collected and stored. At step (708), the processor (308) analyses the received patient data and the details of the surgical procedure to be performed collected at the step (706), with respect to the set of models created at the step (704). At step (710), the processor (308) identifies an appropriate model indicating an optimum placement of ports on the body of the patient.

At step (712), a tracker (310) provided in the cart (300) (as shown in FIG. 3), detects a location of the plurality of robotic carts (SL, PL, PR, SR) each having a surgical instrument (110, 112, 114, 116), with respect to the robotic cart (CA) having the camera (C). The locations of the plurality of robotic carts may be identified with at least a reference point. The reference may be a robotic cart (CA) having the camera (C), an operating table (104), and the like. The reference point is not limited to the reference point as described. In step (714), the graphical user interface (306) displays the location of the plurality of robotic carts (SL, PL, PR, SR). The processor (308) calculates an optimum configuration of each of the robotic arm (102b, 102c, 102d, 102e) and a desired location of each of the plurality of robotic carts (SL, PL, PR, SR), based on the identified model and the detected location of the plurality of robotic carts (SL, PL, PR, SR) in step (716). The plurality of robotic carts (SL, PL, PR, SR) are placed at the desired location with each of the robotic arms (102b, 102c, 102d, 102e) having the optimum configuration in step (718). The robotic surgical carts may be automatically placed to the desired optimum locations or maybe the assistant manually moves the robotic surgical carts. At step (720), the distal end of the plurality of robotic arms (102b, 102c, 102d, 102e) is manipulated, according to the identified optimum placement of ports on the body of the patient. At step (722), the plurality of robotic arms (102b, 102c, 102d, 102e) is docked. At step (724), the plurality of robotic arms (102b, 102c, 102d, 102e) is configured in the best optimum position with respect to a remote centre of motion (RCM) constraints.

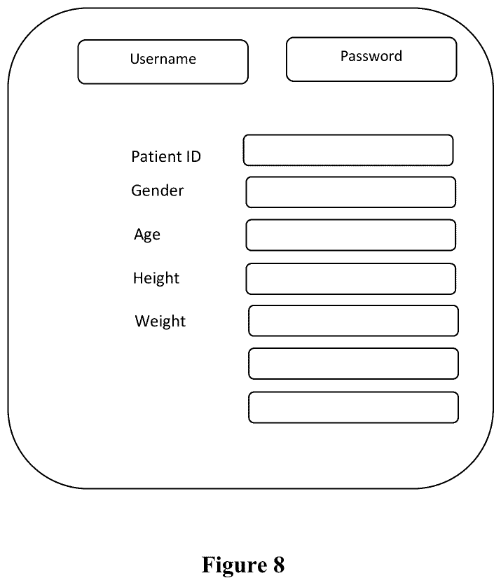

FIG. 8 illustrates an exemplary interface of pre-operative GUI for manually entering patient details in accordance with an embodiment of the disclosure. The pre-operative GUI provides the user with necessary information required for performing preoperative planning to achieve ease of docking and maximum range of motion around remote center of motion (RCM) for arms/instruments all the time while working on surgical area of interest. The pre-operative GUI allows the operator to login to the GUI by entering the provided username and password. After login the operator can manually enter the patient and details of the procedure to be performed. The pre-operative GUI may include the following interfaces such as welcome screen, login screen, home screen, manual or XML screen and main screen. The pre-operative GUI application provides the user with preferred instruments to use, cart positioning, arm docking, patient positions, port placements and a simulation video based on the user selected patient information such as gender, height, weight, number of carts and type of surgery to be performed.

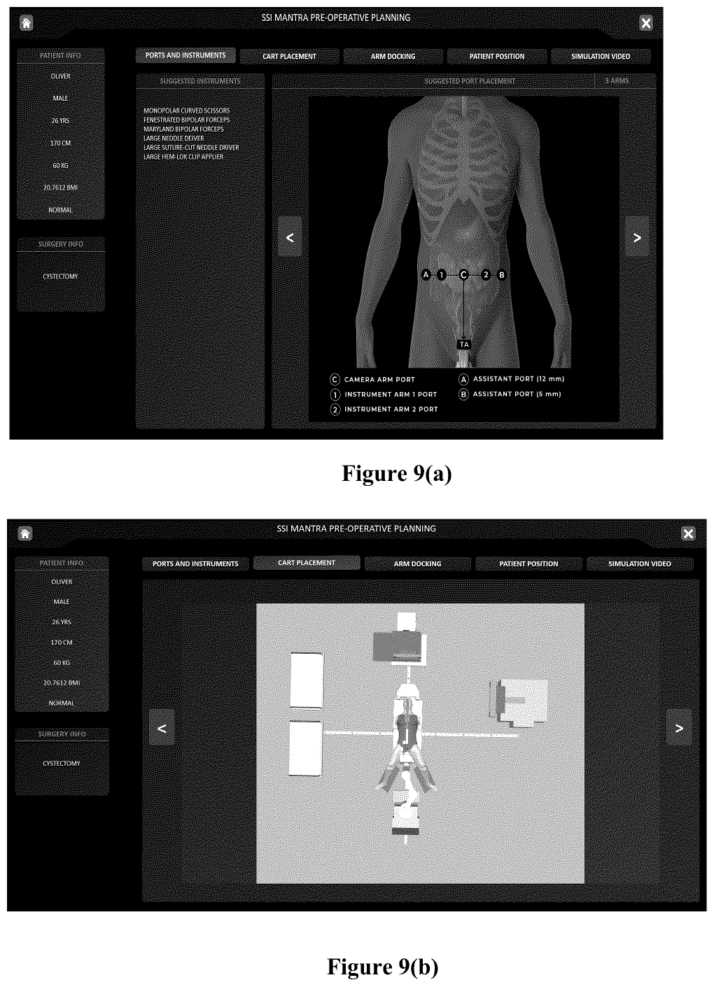

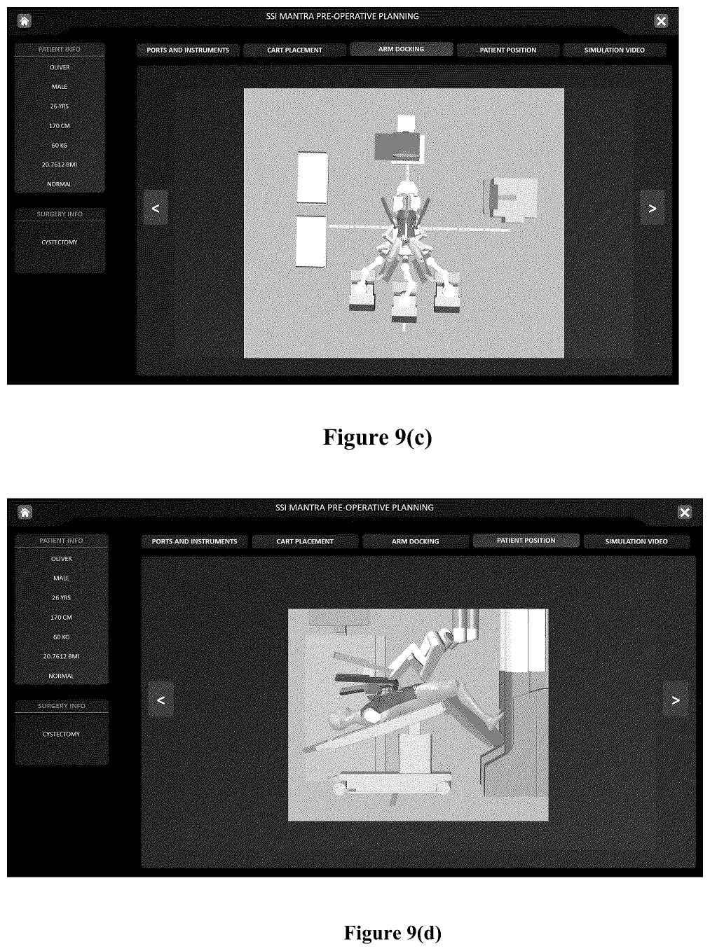

FIG. 9(a) illustrates an exemplary interface of ports and instrument tab of pre-operative GUI for showing suggested instruments and port placements in accordance with an embodiment of the disclosure. FIG. 9(b) illustrates interface of cart placement tab of pre-operative GUI for showing suggested cart placement in accordance with an embodiment of the disclosure. FIG. 9(c) illustrates interface of arm docking tab of pre-operative GUI for showing suggested arm docking in accordance with an embodiment of the disclosure. FIG. 9(d) illustrates interface of patient positioning tab of pre-operative GUI for showing suggested patient positioning in accordance with an embodiment of the disclosure. The pre-operative application can be used for patient data storage and as suggestive pre-operative guidance.

The foregoing description of exemplary embodiments of the present disclosure has been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

List of reference numerals:

| Sr. No. | Component | Reference Numeral(s) |
|---|---|---|
| 1 | Robotic arms | 102a, 102b, 102c, 102d, 102e |
| 2 | Operating table | 104 |
| 3 | Endoscopic camera | C |
| 4 | Surgical instruments | 110, 112, 114, 116 |
| 5 | Server | 302 |
| 6 | Database | 304 |
| 7 | Graphical user interface | 306 |
| 8 | Processor | 308 |
| 9 | Tracker | 310 |
| 10 | Multi-arm robotic surgical | 100 |
| | system | |
| 11 | Robotic cart | SL, PL, CA, PR, SR |

1. A method (700) for pre-operative planning for a multi-arm robotic surgical system (100) comprising a plurality of robotic arms (102a, 102b, 102c, 102d, 102e) each mounted on one of a plurality of robotic carts (SL, PL, CA, PR, SR), an endoscopic camera (C) coupled to a robotic arm (102a) out of the plurality of robotic arms (102a, 102b, 102c, 102d, 102e), a plurality of surgical instruments (110, 112, 114, 116) each detachably coupled to a distal end of a robotic arm out of the remaining robotic arms (102b, 102c, 102d, 102e), an operating table (104), and a patient lying on the operating table (104), whereby the plurality of robotic carts (SL, PL, CA, PR, SR) are arranged along the operating table (104), the method comprising:
storing (702), using a server (302), in a database (304), at least one of various human anatomical data and body habitus;
creating (704), using the server (302), a set of models indicating placement of ports on the body of the patient, based on the stored human anatomical data and body habitus;
receiving (706), using a graphical user interface (306), a required patient data and details of a surgical procedure to be performed;
analysing (708), using a processor (308), the received patient data and details of the surgical procedure to be performed with respect to the set of models;
identifying (710), using the processor (308), an appropriate model out of the set of models indicating an optimum placement of ports on the body of the patient;
detecting (712), using a tracker (310), a location of the plurality of robotic carts (SL, PL, PR, SR) each having a surgical instrument (110, 112, 114, 116), with respect to the robotic cart (CA) having the camera (C);
displaying (714), using the graphical user interface (306), the detected location of the plurality of robotic carts (SL, PL, PR, SR);
calculating (716), using the processor (308), an optimum configuration of each of the robotic arm (102b, 102c, 102d, 102e) and a desired location of each of the plurality of robotic carts (SL, PL, PR, SR), based on the identified model and the detected location of the plurality of robotic carts (SL, PL, PR, SR);
placing (718), the plurality of robotic carts (SL, PL, PR, SR), at the desired location of the plurality of robotic carts (SL, PL, PR, SR) having the optimum configuration of each of the robotic arm (102b, 102c, 102d, 102e);
manipulating (720), the distal end of the plurality of robotic arms (102b, 102c, 102d, 102e) according to the identified optimum placement of ports on the body of the patient;
docking (722), the plurality of robotic arms (102b, 102c, 102d, 102e); and
configuring (724), the plurality of robotic arms (102b, 102c, 102d, 102e), in the best optimum position with respect to a remote centre of motion.

2. The method (700) as claimed in claim 1, wherein the server (302) comprises at least one of a local database (304) or a cloud-based database (314).

3. The method (700) as claimed in claim 1, wherein the patient data may include at least one of anatomy of the patient, name, age, sex, body mass index, height etc.

4. The method (700) as claimed in claim 1, wherein the details of a surgical procedure to be performed may include the name of the procedure.

5. The method (700) as claimed in claim 1, wherein the tracker (310) may be any one of an optical tracker, a laser based tracker, or a RFID based tracker, and like.

6. The method (700) as claimed in claim 1, wherein the tracker (310) can detect the location of the plurality of robotic carts (SL, PL, PR, SR) with respect to at least one of a robotic cart (CA) having the camera (C), an operating table (104), and the like.

7. The method (700) as claimed in claim 1, wherein the tracker (310) sends the detected location of the plurality of robotic carts (SL, PL, PR, SR) to the processor (308).

8. The method (700) as claimed in claim 1, wherein the surgical procedure to be performed may be Prostatectomy, Hysterectomy, Nephrectomy, Coronary artery bypass graft, Mitral valve repair, and the like.

9. The method (700) as claimed in claim 1, wherein the placing of the plurality of robotic carts (SL, PL, PR, SR) at the desired location may be done either automatically or manually.

10. The method (700) as claimed in claim 1, wherein the graphical user interface (306) displays surgical instruments which can be used for the surgical procedure to be performed, position of the patient lying on the operating table (104), and the placement of ports on the body of the patient.

11. The method (700) as claimed in claim 1, wherein the graphical user interface (306) further can display a simulation video based on the received patient data and details of the surgical procedure to be performed.

12. The method (700) as claimed in claim 1, wherein the server is configured to store the patient data and the pre-operative planning for the patient for record keeping and future reference.

13. The method (700) as claimed in claim 1, wherein the patient lying on the operating table (104) can be real or a mannequin used for training purposes.

14. The method (700) as claimed in claim 1, wherein the processor (308) is provided with various parameters to calculate the optimum configuration of each of the robotic arm (102b, 102c, 102d, 102e).

15. The method (700) as claimed in claim 14, wherein the parameters indicate reachability of the plurality of robotic arms (102b, 102c, 102d, 102e), limits of configuration of the plurality of robotic arms (102b, 102c, 102d, 102e), singularities of the plurality of robotic arms (102b, 102c, 102d, 102e), dexterity of the plurality of robotic arms (102b, 102c, 102d, 102e), manipulability of the plurality of robotic arms (102b, 102c, 102d, 102e), and robustness of the plurality of robotic arms (102b, 102c, 102d, 102e).
